(19) United States
(12) Patent Application Publication
(10) Pub. No.: US 2015/0092927 A1
(43) Pub. Date: Apr. 2, 2015

(54) METHOD AND MAGNETIC RESONANCE SYSTEM FOR ACQUIRING MAGNETIC RESONANCE DATA

(71) Applicant: Siemens Aktiengesellschaft, Muenchen (DE)

(72) Inventors: Aurel Jensch, Baiersdorf (DE); Hans-Juergen Mueller, Pretzfeld (DE)

(73) Assignee: SIEMENS AKTIENGESELLSCHAFT, Muenchen (DE)

(21) Appl. No.: 14/501,643

(22) Filed: Sep. 30, 2014

(30) Foreign Application Priority Data
Sep. 30, 2013 (DE) 102013219732.2

(51) Int. Cl. A61B 6/00; A61B 6/03

(52) U.S. Cl. CPC A61B 6/4488; A61B 6/035; A61B 6/4429
USPC 378/199

(57) ABSTRACT

A medical imaging apparatus has a gantry having a stationary gantry housing and a rotor that is rotatable relative to the stationary gantry housing. At least one component to be cooled is arranged at the rotor, and a cooling device produces a cooling of the component. The cooling device has a carrier medium that is ferromagnetic and designed to absorb heat at the rotor and discharge heat to the stationary gantry housing and is transported from the stationary gantry housing to the rotor and back by at least one magnetic field generated by a magnetic field generator.

### BACKGROUND OF THE INVENTION

1. Field of the Invention

The present invention concerns a method to cool a component mounted on a rotor, which rotates within a gantry, of a medical imaging apparatus, as well as a medical imaging apparatus that operates according to such a method.

2. Description of the Prior Art

The gantry of a computed tomography apparatus has an essentially annular stationary part, generally designated as a gantry housing. The gantry housing usually has a fixed support frame on which are attached casing elements or flow plates. A likewise essentially annular part (generally designated as a rotor of the gantry) that rotates in the operation of the computed tomography apparatus is supported on the support frame by a suitable bearing (for example a roller bearing). The rotor has a rotating frame (designated as a drum) on which are attached all rotating components, in particular at least one x-ray radiator as well as at least one x-ray detector that interacts with the x-ray radiator.

In any computed tomography apparatus, the majority of the electrical power required to operate the components (for example the x-ray radiator) is transduced into heat, and only a relatively small portion is transduced into x-ray radiation. For this reason, sufficient cooling is necessary. The components on the rotor of the gantry have conventionally been cooled by air cooling. This takes place by cooling air (most often generated in the stationary part of the gantry) being directed through the components or flowing through an oil/water cooler. The x-ray radiator is most often cooled by an extra cooling circuit, such as warm oil/water being pumped from the x-ray radiator through a heat exchanger. Cool air flows through the heat exchanger; the thermal power loss is thereby passed to the stationary part of the gantry. From there, the air is cooled and sent again into the cooling circuit (water-cooled gantry) or blown into the examination room (air-cooled gantry).

Due to the cooling medium of air that is used for energy exchange between rotor and stationary part of the gantry, large amounts of air are required due to the high power loss of the components of the rotor, or small amounts of air must be moved very quickly. This generally means large ventilators or a large number of ventilator devices, which in turn causes a high noise level. Moreover, air has a relatively low specific heat storage capacity so that large amounts of air are required to discharge the thermal power loss. The air feed (suction, pressure channel, air guide plates, etc.) and the corresponding ventilator devices require a large amount of structural space and a complicated installation in the gantry. Given entirely air-cooled gantries, the warm air is additionally blown into the examination room, which requires a climate control system in these rooms.

Implementing heat transfer by means of a cooling fluid that has a better heat capacity than air (for example water, oil) is very complicated, due to the conduits required for this between the rotor and the stationary gantry housing, or is not practical or is only practical with limitations to the functionality of the rotor.

### SUMMARY OF THE INVENTION

An object of the present invention is to provide a medical imaging apparatus with a gantry which enables an effective and practical cooling of at least one component of the rotor of the gantry; furthermore, it is an object of the invention to provide a method to cool a component of such a medical imaging apparatus.

In the medical imaging apparatus according to the invention, with a gantry having a stationary gantry housing and a rotor rotatable relative to the stationary gantry housing, wherein at least one component to be cooled is mounted on the rotor, the medical imaging apparatus has a cooling device that produces cooling of the component. The cooling device has a carrier medium that is ferromagnetic and designed to absorb heat from the rotor and to discharge heat to the stationary gantry housing. The carrier medium can be transported by at least one magnetic field from the rotor to the stationary gantry housing and back. The apparatus has a device to generate the at least one magnetic field.

In a medical imaging apparatus according to the invention, a particularly effective, compact and silent cooling is possible by the use of a ferromagnetic carrier medium with an optimally high heat capacity and by at least one magnetic field that produces a transport of the carrier medium (and therefore of the heat) without conduits from the rotor to the stationary gantry housing. Since no conduits are required for the transport of the carrier medium, the functionality of the rotor with regard to its rotation is not negatively affected by the cooling device. A faster and more effective cooling than by means of air is ensured by the use of the carrier medium made from iron, steel, nickel or cobalt, all of which have a markedly higher specific heat capacity than air.

Moreover, the invention encompasses a method to cool a component of a medical imaging apparatus on a rotor that rotates in a gantry that includes with the following steps.
The component is cooled by transfer of heat to a ferromagnetic carrier medium, in particular by a cooling element.
At least one magnetic field is controlled to cause transport of the heated carrier medium from the rotor to the stationary gantry housing, across an intervening space between the rotor and the stationary gantry housing.
The heated carrier medium is cooled, in particular an additional cooling element.
The at least one magnetic field is controlled to cause the cooled carrier medium to be transported back to the rotor, across the intervening space.
The at least one magnetic field is generated and controlled so as to be alternatingly polarized in opposite directions for the transport and return transport.

In an embodiment of the invention, the carrier medium adheres to the rotor or to the stationary gantry housing such that it can be released, depending on the magnetic field, and, for example, by a change of the polarity of the magnetic field is transported across the intervening space between the rotor and the stationary gantry housing to the stationary gantry housing, so as to subsequently adhere to the housing such that it can be detached therefrom. The process can be implemented simply and, for example, can be repeated arbitrarily often by a simple polarity reversal of a magnetic field. For example, the magnetic field can be extended from the rotor across the intervening space to the stationary gantry housing. The field lines can be orthogonal to the respective opposing surfaces of the rotor and the stationary gantry housing. For example, the carrier medium can be fashioned in the form of a powder, a granulate or even one or more solid bodies (for example a ring made of steel, matched to the rotor).

According to a further embodiment of the invention, the cooling device has at least one cooling element arranged on the rotor to transfer heat from the component to the carrier medium, and at least one cooling element arranged on the stationary gantry housing to cool said carrier medium. The cooling element on the rotor transports the heat from the component to the carrier medium. For example, this can be a cooling circuit with a cooling fluid, or another device for heat transport. Alternatively, the heat can be transferred directly from the component to the carrier medium. The heated carrier medium is then cooled at the stationary gantry housing in order to be ready to be used again to absorb heat. Any known cooling devices for cooling can be used, for example refrigeration machines. A fluid reservoir can also be provided.

The device to generate the at least one magnetic field is advantageously designed for alternating polarity reversal of the at least one magnetic field, in particular for high-frequency polarity reversal of the magnetic field. A quick transport of heat—and therefore a continuous heat discharge by means of the transport medium—from the rotor to the stationary gantry housing can occur via an optimally fast polarity reversal. An optimally fast cooling of the carrier medium at the stationary gantry housing is required for this.

According to a further embodiment of the invention, the components of an x-ray radiator or an x-ray detector that are to be cooled, and the medical imaging apparatus, are formed by a computed tomography apparatus.

According to a further embodiment of the invention, the medical imaging apparatus has a device to generate two differently polarized magnetic fields that are arranged next to one another, wherein a first magnetic field produces an arrangement of a first portion of the carrier medium at the rotor and a second magnetic field produces an arrangement of a second portion of the carrier medium at the stationary gantry housing. Moreover, a device that produces a transport of the carrier medium along the stationary gantry housing from the second magnetic field to the first magnetic field can be arranged at said stationary gantry housing.

A corresponding method during a rotation of the rotor includes the following steps. Two oppositely polarized magnetic fields situated next to one another are controlled to cause transport of a first portion of the heated ferromagnetic carrier medium from the rotor to the stationary gantry housing via the first magnetic field, and to cause transport of a second portion of the cooled carrier medium from the stationary gantry housing to the rotor takes place via the second magnetic field. The second part of the carrier medium is transported along the stationary gantry housing from the second magnetic field to the first magnetic field.

A continuous cooling process can be produced during the rotation. For example, one realization is such that a first magnetic field is arranged in the region of the first half of the rotor ring (for example a semicircle, thus encompassing 180°) and of the opposite stationary gantry housing, and a second magnetic field that is directed opposite the first magnetic field is arranged in the region of the second half of the rotor ring and the opposite stationary gantry housing. The first magnetic field produces the transport (or the continued position) of the second portion of a first portion of the carrier medium at the rotor, while the second magnetic field produces the transport (or the continued position) of the second portion of the carrier medium at the stationary gantry housing. By the rotation of the rotor, the first portion of the carrier medium moves into the region of the second magnetic field and is thereby transported to the stationary gantry housing, and the therefore to the second portion of the carrier medium. Since no rotation occurs at the stationary gantry housing, the original second portion must be transported via a special device into the region of the first magnetic field in order to be transported to the rotor (and therefore to the first portion). This transport movement is likewise implemented in the rotation direction of the rotor, for example.

According to a further embodiment, the medical imaging apparatus has a device to generate a third magnetic field which produces the transport of the carrier medium along the stationary gantry housing from the second magnetic field to the first magnetic field. For example, such a third magnetic field is situated at the stationary gantry housing such that it moves the carrier medium to the stationary gantry housing in the inflow region of the first magnetic field, for example along the surface of said stationary gantry housing. The third magnetic field can itself be moved, for example, or can vary in its strength in order to produce the movement. For example, the movement can be designed similar to the rotation movement of the rotor. For example, the third magnetic field can be arranged on the stationary gantry housing behind a non-magnetic layer.

Alternatively, a transport of the carrier medium in the region of the first magnetic field can also be produced by a device with a cooling fluid, for example via pumps or coils. A cooling of the carrier medium can additionally take place via the cooling fluid.

### BRIEF DESCRIPTION OF THE DRAWINGS

FIG. 1 is a side view of a medical imaging apparatus according to the invention with: a rotor; a stationary gantry housing; a carrier medium in the form of a granulate; and a magnetic field with a first polarity.

FIG. 2 is a side view of the medical imaging apparatus according to the invention according to FIG. 1 with a second polarity of the magnetic field.

FIG. 3 is a side view of a medical imaging apparatus according to the invention with a carrier medium in the form of a powder.

FIG. 4 is a side view of a medical imaging apparatus according to the invention with a carrier medium in the form of a solid ring.

FIG. 5 is a side view of a medical imaging apparatus according to the invention with: a rotor; a stationary gantry housing; a carrier medium in the form of a granulate; and two oppositely polarized magnetic fields.

FIG. 6 is a front view of the surface of the rotor of the medical imaging apparatus according to FIG. 5, which surface faces towards the stationary gantry housing.

FIG. 7 is a front view of the surface of the stationary gantry housing of the medical imaging apparatus according to FIG. 5, which surface faces towards the rotor.

FIG. 8 shows a sequence of a method according to the invention for cooling a component of a medical imaging apparatus.

### DESCRIPTION OF THE PREFERRED EMBODIMENTS

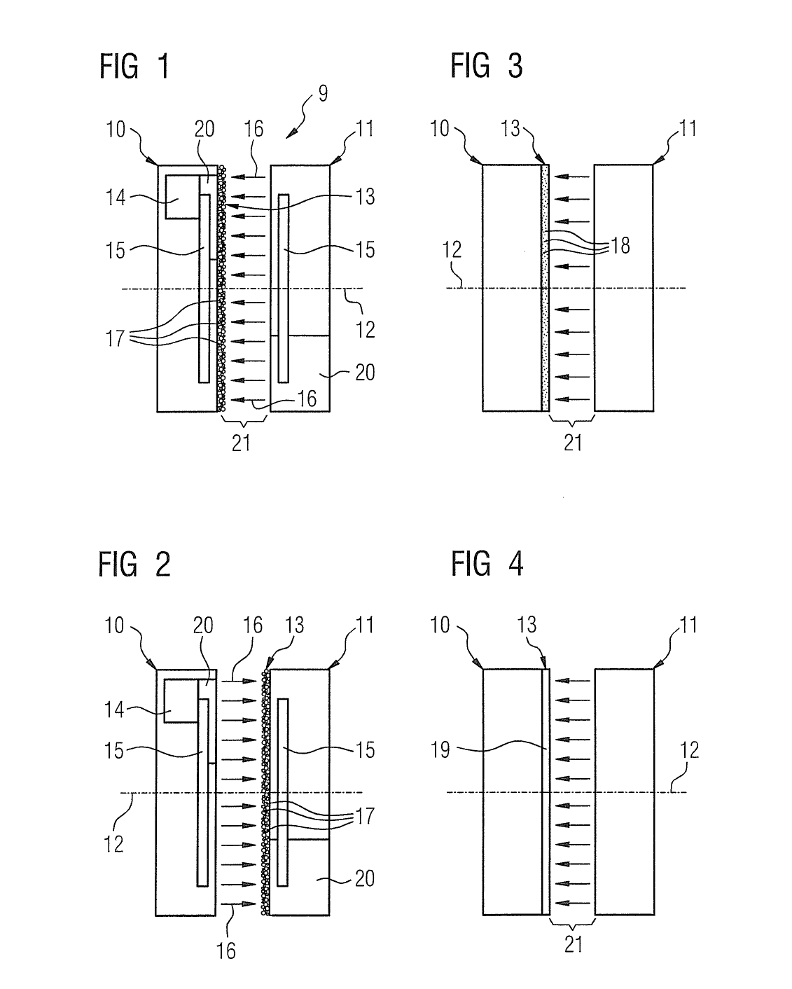

A side view of a medical imaging apparatus according to the invention in the form of a computed tomography apparatus with a gantry 9 is shown in FIG. 1, wherein the gantry 9 has an annular rotor 10 which is designed for a rotation around an axis 12 and a stationary gantry housing 11 (also called a stator). For example, an x-ray radiator 14 and an x-ray detector (not shown) are arranged on the rotor 10. Given a rotation of the rotor 10 around a patient arranged in the center of the ring and in the region of the axis 12, x-rays are emitted by the x-ray radiator 14, which x-rays penetrate the patient and are acquired by the x-ray detector in a form that is attenuated in a manner characteristic of the patient. A significant heating occurs during the operation of the x-ray radiator, such that a cooling of the radiator (and possibly of additional components, for example the x-ray detector) is necessary.

The release heat must be transported away from the rotor as effectively as possible. For this, on the one hand a carrier medium 13 is provided which has an optimally high heat capacity and can simultaneously be transported via an at least one magnetic field across an intervening space 21 between the rotor 10 and the stationary gantry housing (thus is ferromagnetic). For example, the carrier medium 13 can be formed by a granulate 17 made of iron and/or stored. Alternatively, as shown in FIGS. 3 and 4 a powder 18 or one or more solid bodies 19 can also be used. For example, the solid body 19 can be formed by a steel ring which has a shape adapted to the rotor 10.

A magnetic field, a magnetic field generator 15 generates at least one magnetic field, the direction of which is indicated by arrows 16 in the intervening space 21 between the rotor 10 and the stationary gantry housing 11. For example, the magnetic field can extend over the entire ring of the rotor 10 (as shown) or can also cover only a portion of this. In FIG. 1, the magnetic field is polarized such that the ferromagnetic carrier medium 13 adheres to the rotor. Via a change of the magnetic field (for example by a polarity reversal of the direction of the magnetic field) the carrier medium 13 (thus for example the granulate 17 made of iron) is transported across the intervening space 21 to the stationary gantry housing 11 and adheres there. The magnetic field generator 15 can be arranged at the gantry (thus on the rotor and/or the stationary gantry housing, for example); however, it can also be arranged outside of the gantry. The magnetic field generator 15 can be formed by one or more coils, for example. Alternatively, the magnetic field can be formed on one side by one or more permanent magnets, and the polarity reversal is achieved via an electrically generated magnetic field. The electrical magnetic field thus can be simply superimposed on that of the permanent magnet.

A method to cool the x-ray radiator 14 can proceed as shown in FIG. 8, as an example. The magnetic field is initially set such that the carrier medium 13 is located at the rotor, in particular in the region of the surface of the rotor 10 that faces towards the stationary gantry housing, and adheres there. This situation is shown as an example in FIG. 1. In a first step 25, heat is transferred from the x-ray radiator 14 (or the other component) to the carrier medium 13 in order to cool the x-ray radiator. For example, this can be implemented by means of a cooling element 20 located between the x-ray radiator 14 and the carrier medium. The cooling element 20 can be formed by a cooling circuit or other cooling device, for example. A direct heat transfer can also be implemented, for example via a corresponding arrangement of the x-ray radiator 14. In a second step 26, the heated carrier medium 13 is transported across the intervening space 21 to the stationary gantry housing 11 via a corresponding control of the magnetic field (for example a polarity reversal in the opposite direction than the previous one) so that said carrier medium 13 then adheres to, for example, the surface of the stationary gantry housing 11 that faces towards the rotor 10. This situation is shown in FIG. 2, for example.

The heated carrier medium 13 is now cooled in a third step 27. This can take place solely via a cooler surface of the stationary gantry housing, or via one or more additional cooling elements 20 or cooling devices or, respectively, refrigeration machines. The cooled carrier medium 13 is subsequently transported back to the surface of the rotor 10 in a fourth step via control of the magnetic field (for example polarity reversal in the opposite direction again). The method can be repeated continuously, for example during the operation of the x-ray radiator, such that a continuous heat flow takes place from the rotor to the stationary gantry housing. The switching of the magnetic field can also take place very quickly—thus at high frequency, for example—in particular given use of granulate or powder.

For example, the magnetic field can extend over the entire ring of the rotor (as shown) or also cover only a portion of this. Multiple magnetic fields (for example four) can also be present in multiple segments of the rotor, of which two respectively have opposite polarity so that half of the carrier medium respectively adheres to the rotor and half adheres to the stationary gantry housing. In coordination with the rotation of the rotor (for example thus after a 360° rotation), the polarity of these magnetic fields is then likewise reversed. Many additional embodiments of the invention are also conceivable.

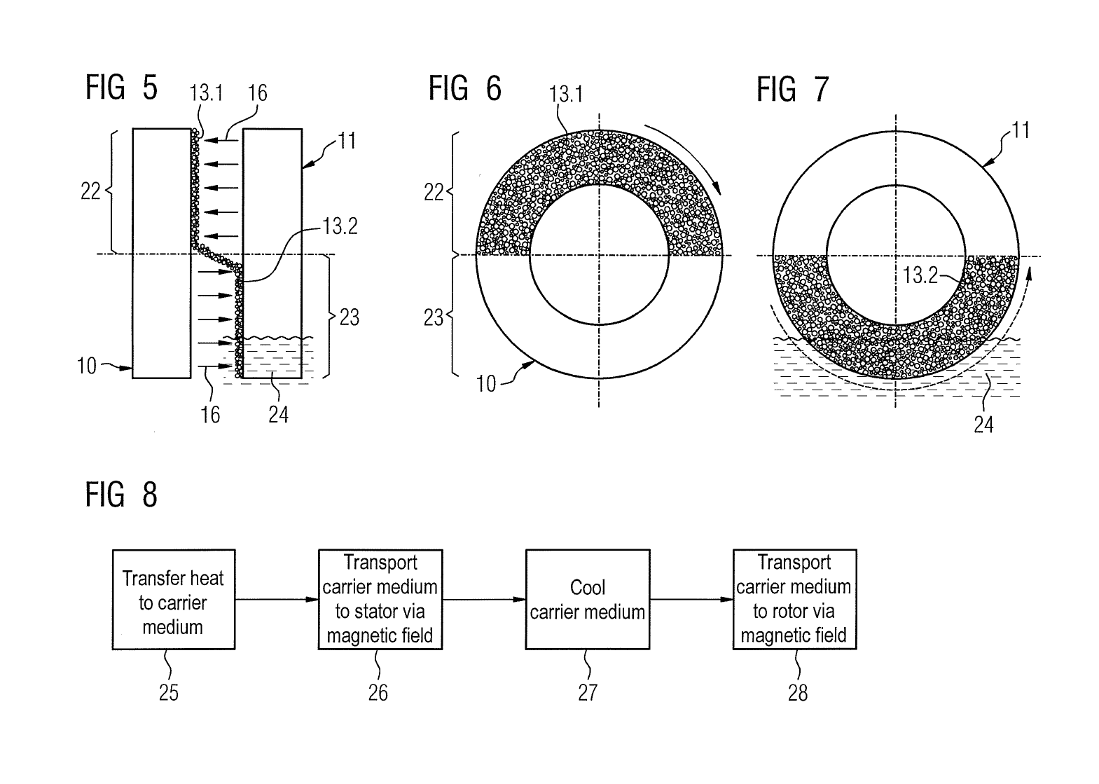

A further embodiment of the invention is shown in FIGS. 5 through 7, in which two opposite, fixed magnetic fields are present next to one another which are not modified. In FIG. 5 a side view is shown, while in FIG. 6 a plan view of the surface of the rotor 10 situated opposite the stationary gantry housing 11 is shown, and in FIG. 7 a plan view is shown of the surface of the stationary gantry housing 11 that is situated opposite the rotor 10.

As shown in FIG. 5, a first magnetic field 22 is arranged and controlled in (for example) the region of the half ring of the rotor and accordingly across the intervening space up to the stationary gantry housing, wherein the first magnetic field 22 is initially produced such that a first portion of the carrier medium 13.1 adheres to the rotor 10. It must be ensured that the first magnetic field 22 is also stationary given a rotation of the rotor. A second magnetic field 23 is arranged and controlled in the region of the other half of the ring of the rotor and accordingly across the intervening space up to the stationary gantry housing, such that initially a second portion of the carrier medium 13.2 adheres to the stationary gantry housing 11. If the rotor 10 now rotates, given stationary magnetic fields the first portion of the carrier medium 13.1 that is located at the rotor comes, after a maximum of a 180° rotation, into the region of the second magnetic field 23 and is transported to the stationary gantry housing. In return, the second portion of the carrier medium 13.2 that is located at the stationary gantry housing 11 must be transported along the surface of said stationary gantry housing 11 into the region of the first magnetic field 22. For example, this can be implemented via a fluid 24 which, for example, is arranged in a container and is accordingly pumped or moved. Alternatively, a third magnetic field that is limited in its extent to the stationary gantry housing can also be used for the transport along the surface of said stationary gantry housing. For this, a “wandering” magnetic field—thus a magnetic field that either varies in its strength or that itself is moved—can be applied behind a non-ferromagnetic plate on the surface of the stationary gantry housing. In which direction the transport of the carrier medium preferably takes place (dashed arrow) in order to correspond to the rotation (arrow) shown in FIG. 6 is shown in FIG. 7. A cooling via the cooling fluid 24 can likewise be present.

The invention describes a method and a device by means of heat is bound to a material, which material can be moved via magnetic fields; stated in a different way, heat can be made magnetic and transmitted. The advantage lies in a nearly silent, compact and—due to the high specific heat capacity—particularly effective heat transfer, and therefore cooling.

The invention can be briefly summarized as follows: a medical imaging apparatus is provided, with a gantry having a stationary gantry housing and a rotor rotatable relative to the stationary gantry housing, wherein at least one component to be cooled is arranged at the rotor, wherein the medical imaging apparatus has: a cooling device, which cooling device produces a cooling of the component, wherein the cooling device has: a carrier medium which is ferromagnetic and is designed to absorb heat at the rotor and to discharge heat to the stationary gantry housing and can be transported from the stationary gantry housing to the rotor and back by means of at least one of magnetic field; and a device to generate the at least one magnetic field.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical imaging apparatus comprising:
a gantry comprising a stationary gantry housing and a rotor that is rotatable in and relative to the stationary gantry housing;
at least one apparatus component mounted on said rotor, that produces heat during operation thereof;
a cooling device comprising a ferromagnetic carrier medium; and
a magnetic field generator that generates at least one magnetic field that interacts with said ferromagnetic carrier medium to transport said ferromagnetic carrier medium into thermal communication with said apparatus component so as to absorb said heat into said ferromagnetic carrier medium, and to transport said ferromagnetic carrier medium from said apparatus component to said stationary gantry housing to discharge said heat from the ferromagnetic carrier medium to the stationary housing.

2. A medical imaging apparatus as claimed in claim 1 wherein said ferromagnetic carrier medium is comprised of a material that releasably adheres to said rotor or to said stationary gantry housing, dependent on said at least one magnetic field, and wherein said at least one magnetic field generated by said magnetic field generator transports said ferromagnetic carrier medium across an intervening space between said rotor and said stationary gantry housing by changing a polarity of said at least one magnetic field.

3. A medical imaging apparatus as claimed in claim 1 wherein said ferromagnetic carrier medium is a powder or granulate material.

4. A medical imaging apparatus as claimed in claim 3 wherein said ferromagnetic carrier medium is at least partially formed of material selected from the group consisting of iron, steel, nickel and cobalt.

5. A medical imaging apparatus as claimed in claim 1 wherein said cooling device comprises a cooling element situated at said rotor that transfers heat from said apparatus component to said ferromagnetic carrier medium, and at least one further cooling element situated at said stationary gantry housing that removes heat from said ferromagnetic carrier medium.

6. A medical imaging apparatus as claimed in claim 1 wherein said magnetic field generator generates said at least one magnetic field with alternating polarity reversal.

7. A medical imaging apparatus as claimed in claim 1 wherein said apparatus component is selected from the group consisting of an x-ray radiator and an x-ray detector.

8. A medical imaging apparatus as claimed in claim 1 wherein said magnetic field generator generates two differently polarized magnetic fields next to one another, with a first of said magnetic fields producing an arrangement of a first portion of said ferromagnetic carrier medium at said rotor, and a second of said magnetic fields producing an arrangement of a second portion of said ferromagnetic carrier medium at said stationary gantry housing.

9. A medical imaging apparatus as claimed in claim 8 comprising a transport device at said stationary gantry housing that transports said second portion of said ferromagnetic carrier medium along said stationary gantry housing from said second of said magnetic fields to said first of said magnetic fields.

10. A medical imaging apparatus as claimed in claim 9 wherein said magnetic field generator generates a third magnetic field that transports said second portion of said ferromagnetic carrier medium along said stationary gantry housing from said second of said magnetic fields to said first of said magnetic fields.

11. A medical imaging apparatus as claimed in claim 9 comprising a cooling fluid transport device that transports said second portion of said ferromagnetic carrier medium along said stationary gantry housing from said second of said magnetic fields to said first of said magnetic fields.

12. A method for cooling a medical imaging apparatus comprising a gantry comprising a stationary gantry housing and a rotor that is rotatable in and relative to the stationary gantry housing, and at least one apparatus component mounted on said rotor, that produces heat during operation thereof, said method comprising:
cooling said apparatus component using a ferromagnetic carrier medium; and
generating at least one magnetic field that interacts with said ferromagnetic carrier medium to transport said ferromagnetic carrier medium into thermal communication with said apparatus component so as to absorb said heat into said ferromagnetic carrier medium, and to transport said ferromagnetic carrier medium from said apparatus component to said stationary gantry housing to discharge said heat from the ferromagnetic carrier medium to the stationary housing.

13. A method as claimed in claim 12 comprising using, as said ferromagnetic carrier medium, a material that releasably adheres to said rotor or to said stationary gantry housing, dependent on said at least one magnetic field, and with said at least one magnetic field, transporting said ferromagnetic carrier medium across an intervening space between said rotor and said stationary gantry housing by changing a polarity of said at least one magnetic field.

14. A method as claimed in claim 12 comprising, with a cooling element situated at said rotor, transferring heat from said apparatus component to said ferromagnetic carrier medium, and with at least one further cooling element situated at said stationary gantry housing, removing heat from said ferromagnetic carrier medium.

15. A method as claimed in claim 12 comprising generating said at least one magnetic field with alternating polarity reversal.

16. A method as claimed in claim 12 comprising generating two differently polarized magnetic fields next to one another, and with a first of said magnetic fields, producing an arrangement of a first portion of said ferromagnetic carrier medium at said rotor, and with a second of said magnetic fields, producing an arrangement of a second portion of said ferromagnetic carrier medium at said stationary gantry housing.

17. A method as claimed in claim 16 comprising generating a third magnetic field and, with said third magnetic field, transporting said second portion of said ferromagnetic carrier medium along said stationary gantry housing from said second of said magnetic fields to said first of said magnetic fields.
